(19) United States
(12) Patent Application Publication
(10) Pub. No.: US 2023/0173135 A1
(43) Pub. Date: Jun. 8, 2023

(54) PROCESS TO COAT A MEDICAL DEVICE SURFACE WITH PEPTIDE-BASED NANOPARTICLES

(71) Applicants: UNIVERSIDADE DE COIMBRA, Coimbra (PT); CENTRO DE NEUROCIENCIAS E BIOLOGIA CELULAR, Coimbra (PT)

(72) Inventors: Akhilesh RAI, Coimbra (PT); Lino DA SILVA FERREIRA, Condeixa-A-Nova (PT)

(21) Appl. No.: 17/905,690

(22) PCT Filed: Mar. 10, 2021
(86) PCT No.: PCT/IB2021/051999

§ 371 Date: Sep. 6, 2022

(30) Foreign Application Priority Data
Mar. 10, 2020 (PT) 116154

(51) Int. Cl. A61L 15/44; A61L 15/26; A61L 15/58

(52) U.S. Cl. CPC A61L 15/44; A61L 15/26; A61L 15/58; A61L 2300/252; A61L 2300/404; A61L 2300/412; A61L 2300/608; A61L 2300/624; A61L 2420/02

(57) ABSTRACT

A process of coating a medical device surface with peptide-based nanoparticles with antimicrobial and healing properties; a process to coat a polyurethane (PU) dressing with a cross-linkable polymer adhesive in which was immobilized LL37 peptide conjugated-gold (Au) nanoparticles (LL37NPs) suitable to be applied on wounds. by following the steps of: 1) preparation of medical device surface; 2) coating the surface with a cross-linkable polymer adhesive; 3) spreading of peptide-based nanoparticles over the surface coated with the photo cross-linkable polymer adhesive; 4) exposing the surface coated with the adhesive and the nanoparticles to UV light; 5) placing the surface in phosphate buffer to leach loosely bound nanoparticles. The process described herein may be employed in the production of wound dressings, bandages, PU catheters and medical tubings.

### TECHNICAL FIELD

This application relates to a process of coating a medical device surface with peptide-based nanoparticles with antimicrobial and healing properties. Preferably, the present application relates to a process to coat a polyurethane (PU) dressing with an adhesive in which was immobilized LL37-Au conjugated peptide-gold nanoparticles (LL37NPs) suitable to be applied on wounds.

### BACKGROUND ART

Chronic wounds affect more than 6 million patients in the United States and represent an annual cost of approximately $30 billion[1]. Chronic wounds generally fail to heal within timely manner (up to 12 weeks) in comparison with normal wounds because the regenerative processes are impaired and they are more susceptible to infections. Indeed, wound infections are one of the major factors for delayed healing of chronic wounds due to inhibition of re-epithelialization and collagen production. Conventional wound management involving surgical debridement, swabbing cleaning, dressings and systemic antibiotics do not always yield satisfactory skin healing[2]. The acceleration of wound healing is an objective targeted for a long time in chronic wounds. So far, there only exists a product to accelerate wound healing approved by the FDA[3]. Becaplermin gel 0.01% (Regranex), recombinant human platelet-derived growth factor (PDGF) is produced through genetic engineering and was approved by the US Food and Drug Administration (FDA) in 1997 to promote healing in chronic lower extremity diabetic neuropathic ulcers. The therapy is indicated for uninfected diabetic foot ulcers. PDGF is a mitogen of fibroblasts and smooth muscle cells; it induces chemotaxis of skin cells that migrate from the vicinity of the wound to the wound bed, and stimulates the synthesis of extracellular matrix by the cells. Unfortunately, this product is expensive and is only approved for a specific chronic wound type e.g., diabetic neuropathic ulcers and not for infected wounds. In addition, in some antimicrobial dressings, the leaching of Ag ions and Ag NPs from wound dressings can induce bacteria resistance, allergic reactions, permanent pigmentation and toxic effects in the kidney and liver[4]. Therefore, it is critical the development of wound dressings having simultaneously antimicrobial and healing properties.

### SUMMARY

This application relates to a process of coating a medical device surface with peptide-based nanoparticles with antimicrobial and healing properties.

The present patent application discloses the process of coating a medical device surface comprising the steps of:
Preparation of a medical device surface;
Coating the medical device surface with a photo cross-linkable polymer adhesive;
Immobilization of peptide-based nanoparticles over the top of the surface coated with the cross-linkable polymer adhesive after UV curing;
Exposing the surface coated with the cross-linkable polymer adhesive and peptide-based nanoparticles to an UV light source with wavelength of from 365 to 395 nm;
Placing the medical device surface in phosphate buffer at a pH between 6 and 7.5 to leach loosely bound nanoparticles.

In one embodiment, the medical device surface is placed in the phosphate buffer for 120 to 360 minutes.

In one embodiment, the cross-linkable polymer adhesive has a viscosity between 3 and 300 cP.

In one embodiment, the medical device surface comprises a film selected from polyurethane (PU), polystyrene (PS), poly(ethylene terephthalate) (PET) and polycarbonate (PC).

In one preferable embodiment, the medical device is a wound dressing comprising a polyurethane (PU) film.

In one embodiment, the cross-linkable polymer adhesive is a photo cross-linkable adhesive.

In one preferable embodiment, the photo cross-linkable polymer adhesive is selected from acrylated epoxies, acrylated polyesters, vinyl ethers, N-vinyl compound and vinylpyrrolidone compounds.

In one embodiment, the cross-linkable polymer adhesive is a non-photo cross-linkable adhesive.

In one embodiment, the non-photo cross-linkable polymer adhesive is selected from dopamine, polyethylenimine, amino-propyltrimethoxy silane, polymer brushes containing trifluromethacrylate and 2hydroxyethyl methacrylate.

In one preferably embodiment, the peptide-based nanoparticles are LL37 NPs, wherein said peptide is LL37 (SEQ ID NO:1).

In one embodiment, the LL37 NPs are solubilized in ethanol, acetone, and dimethoxy sulfoxide (DMSO).

In one embodiment, the distance between UV light source and the film should be between 6 to 8 cm in order to coat LL37NPs.

In one embodiment, the amount of polymer adhesive should be between 10 to 30 µL per cm² of film surface.

In one embodiment, the LL37 NPs have a concentration of 40 to 70 µg NPs/cm².

In one embodiment, the amount of peptide available on the surface of medical device should be between 13 to 23 µg / cm2.

The present patent application also discloses a medical device comprising a medical device surface, a photo cross-linkable polymer adhesive, a cross-linkable polymer adhesive and LL37 NPs, wherein said peptide is LL37 (SEQ ID NO: 1).

In one embodiment, the cross-linkable polymer adhesive has a viscosity of between 3 and 300 cP.

In one embodiment, the medical device surface comprises a film selected from polyurethane (PU), polystyrene (PS), poly(ethylene terephthalate) (PET) and polycarbonate (PC).

In one preferable embodiment, the medical device is a wound dressing comprising a polyurethane (PU) film.

In one embodiment, the cross-linkable polymer adhesive is a photo cross-linkable polymer adhesive.

In one preferable embodiment, the photo cross-linkable polymer adhesive is selected from acrylated epoxies, acrylated polyesters, vinyl ethers, N-vinyl compound and vinylpyrrolidone compounds.

In one embodiment, the cross-linkable polymer adhesive is a non-photo cross-linkable polymer adhesive.

In one preferable embodiment, the non-photo cross-linkable polymer adhesive is selected from dopamine, polyethylenimine, amino-propyltrimethoxy silane, polymer brushes containing trifluromethacrylate and 2hydroxyethyl methacrylate.

In one preferable embodiment, the LL37 NPs are immobilized on the top of the cross-linkable polymer adhesive coating the medical device.

In one embodiment, the medical device has a water contact angle lower than 60°.

In one embodiment, the medical device surface comprises 10 to 30 µL per cm² of film surface.

In one embodiment, the LL37 NPs have a concentration of 40 to 70 µg NPs/cm².

### DESCRIPTION OF THE INVENTION

The present patent application relates to a process of coating a medical device surface with peptide-based nanoparticles with antimicrobial and healing properties. Preferably, the present application relates to a process to coat a polyurethane (PU) dressing with an adhesive in which was immobilized LL37 peptide conjugated-gold (Au) nanoparticles (LL37NPs) suitable to be applied on wounds.

In the approach disclosed in the present application, photo cross-linkable polymer adhesive has been used to coat antimicrobial peptide containing nanoparticles on a medical device surface, such as a wound dressing. Polymer adhesive polymerize under the exposure of UV light (wavelength 365 nm), which in turn immobilize the LL37NPs present on the top of them. The advantage of current method is that there is non-significant leaching of the coated peptide-nanoparticle conjugates from the wound dressing in solution or in wound environment, yet show antimicrobial and skin regeneration properties. However, other commercially available antimicrobial wound dressings release a significant amount of silver ions (Ag⁺).

Particularly, photo cross-linkable polymer adhesive has been used to coat antimicrobial peptide containing nanoparticles (LL37NPs) on polyurethane (PU) film. Polymer adhesive polymerize under the exposure of UV light (wavelength 365 nm), which in turn immobilize LL37NPs present on the top of said film. 40 to 70 µg of LL37NPs can be coated on 1 cm² PU film, which, in a preferable embodiment corresponds to 13 to 23 µg of LL37 peptide. The distance between UV light and PU films should be between 6 to 8 cm in order to have strong coating of LL37NPs. Importantly; LL37NPs are synthesized using 0.25 mM LL37 peptide and 0.5 mM HAuCl₄ in the presence of HEPES buffer of pH 5 and 7.5. The advantage of current method is that there is non-significant leaching of the coated LL37NPs from the PU film in solution or in wound environment, yet show antimicrobial and skin regeneration properties. However, other commercially available antimicrobial wound dressings release a significant amount of silver ions (Ag⁺). To the best of the applicant’s knowledge, no reports have been shown to use photo cross-linkable polymer adhesive to coat antimicrobial peptide-nanoparticles on the surface of a medical device such as a wound dressing, bandages, medical tubing and PU catheters.

Therefore, it is herein disclosed a method to produce a dressing that has the ability to promote wound healing and simultaneously prevent microbial infections. The dressing is composed by a polyurethane (PU) film coated with an adhesive in which was immobilized LL37 peptide conjugated gold (Au) nanoparticles (LL37NPs) (from now on named as PU-adhesive-LL37NPs) (FIG. 2). In order to validate the advantages provided by this approach, morphological characterization of PU-adhesive-LL37NPs was performed followed by antibacterial activity against gram-positive and gram-negative bacteria in human serum. Additionally, wound healing potential of the dressing in a diabetic mouse full thickness excisional model is also evaluated. PU-adhesive-LL37NP dressing induces the expression of keratin along with recruitment of macrophage in wounds. Overall results disclosed herein show that PU-adhesive-LL37NP dressing has enhanced antimicrobial activity and superior skin regeneration properties than PU dressing.

### Materials and Methods

Preparation of LL37NPs. LL37 peptide modified with a C-terminal cysteine amino acid, LLGDFFRKSKEKIGKEFKRIVQRIKDFLRNLVPRTESC (SEQ ID NO: 1), was purchased from Caslo Laboratory, Denmark. The purity of the peptide was 96%. LL37NPs were prepared in a one-step approach as described previously in[10]. Briefly, LL37-SH peptide (0.5 mM) was dissolved in DMF (100 µL) followed by addition of HEPES buffer (900 µL, 100 mM, pH 5). To prepare NPs, a LL37 solution (900 µL, 0.25 mM) was added to HAuCl₄·3H₂O (50 µL, 0.01 M, Sigma-Aldrich) solution and then incubated at 25° C. for 24 h. The synthesized LL37-Au NPs were centrifuged at 11.000 rpm for 15 min at 4° C. The collected pellet was resuspended in an absolute ethanol to make 6 mg/mL of stock solution and kept in fridge until used.

Fabrication of PU-Adhesive-LL37NP dressing. To prepare PU-Adhesive-LL37NP dressings, MSK-111 adhesive (10 µL, Dymax) was uniformly coated on 1 cm² PU film (DelStar Technologies, U.K) using a blade spreader and immediately LL37NPs (15 µL, 6 mg/mL dissolved in ethanol) were spared on 1 cm². The coated PU films were placed under UV lamp (365 nm) for 4 min in order to polymerize the adhesive and immobilize the NPs on the surface of PU film. The distance between UV lamp and PU films should be between 6 to 8 cm in order to have effective polymerization of polymer adhesive. Finally, the PU-Adhesive-LL37NP dressing was placed in 100 mM phosphate buffer (pH 7.2) for 2 h to leach loosely bound Au NPs.

Quantification of LL37NPs from PU-Adhesive-LL37NP dressings and the leached solution. PU-Adhesive-LL37NP dressing and leached solutions were digested with nitric acid. After acidic digestion, samples were diluted to 4 mL in milli-Q water and gold was quantified by inductively coupled plasma-mass spectroscopy (ICP-MS), using a Bruckner 820-MS instrument (Fremont, CA, USA). Elemental analysis detection of Au¹⁹⁷ was performed after a calibration of the apparatus using gold (Panreac) as standard at 5, 10, 50, 100, 250, 500 and 1,000 µg/L. Iridium (Panreac) was used as internal standard at 20 µg/L. Data analysis was performed in order to express the amount of NPs (µg) per cm² of PU-Adhesive-LL37NP dressing.

Zeta potential measurements. The zeta potential measurements of the PU-Adhesive-LL37NP and PU dressings was performed using an Electro Kinetic Analyzer (EKA) equipment with stamp cell and RTU titration unit (Anton Paar Gmbh, Graz, Austria). Streaming potential and KCl 1 mM as electrolyte was used. The zeta potential instrument was calibrated with 3 standards, such as pH 4, 7 and 10 prior to the sample analysis. 400 mbar pressure was maintained and six measurements were performed for each sample. All analyses were performed at room temperature.

X-ray photoemission spectroscopy (XPS) analyses. XPS analyses of PU-Adhesive-LL37NP and PU dressings were performed using a ESCALAB 200A, VG Scientific (UK) with PISCES software for data acquisition and analysis. For XPS analysis, an achromatic Al (Ka) X-ray source (1486.6 eV) operating at 15 kV (300 W) was used. The spectrometer was calibrated with reference to Ag 3d_{5/2} (368.27 eV). The XPS spectrometer was operated in CAE mode with pass energy of 20 eV (ROI) and 50 eV (survey). Data acquisition was performed with a pressure lower them 10⁻⁶ Pa. The effect of the electric charge was corrected by the reference of the carbon peak (285 eV). The deconvolution of spectra was performed using the XPSPEAK41 program, in which an adjustment of the peaks was performed using peak fitting with Gaussian-Lorentzian peak shape and Shirley type background subtraction.

Fourier transformed Infrared (FTIR) analyses. The PU-Adhesive-LL37NP and PU dressings were characterized using a golden gate attenuated total reflection (ATR) accessory in a PerkinElmer spectrophotometer. The PU-Adhesive-LL37NP and PU dressings were dried completely before FTIR measurements. Spectra were recorded with 64 scans at resolution of 4 cm⁻¹ being average and then smoothed by 11 points adjacent averaging.

Water contact angle measurements. Contact angle measurements of PU-Adhesive-LL37NP and PU dressings were performed using the sessile drop method with a contact angle measuring system from Data Physics, model OCA 15, equipped with a video CCD-camera and SCA 20 software. PU-Adhesive-LL37NP and PU dressings (2 cm²) were placed in a closed, thermostated chamber (25° C.), and then a water droplet (5 µL) was added on both surfaces with an electronically regulated syringe. The advancing contact angle was determined from three samples.

Antimicrobial activity test. Gram positive S. (aureus) and Gram negative E. (coli and P.aeruginosa) were grown in TSY media for 12 h at 37° C. Bacterial suspensions (100 µL) were transferred to sterile vials containing TSY media (5 mL) and incubated for 2 h at 37° C. to get mid logarithmic phase growth of bacteria then further diluted using 10% human serum (HS) to achieve 5 × 10⁵ CFU/mL bacteria. To test the antimicrobial activity of PU-Adhesive-LL37NP and PU dressings, S.aureus, E.coli, and P.aeruginosa suspensions (100 µL) were placed on the top of the dressings and the sterile parafilm was placed on the top of bacterial suspension in order to spread bacterial suspensions all over the dressings. Then, samples were placed in a humidified chamber and kept at 37° C. for 18 h. After 18 h bacterial suspensions were taken out from PU-Adhesive-LL37NP and PU dressings and serially diluted in PBS and then plated on TSY agar plates followed by incubation at 37° C. Bacterial colonies grown on plates were counted. All antibacterial activity tests in human serum were performed in triplicate to verify the reproducibility of the data.

Bacterial resistance assay. Commercial available Acticoat® dressing from Smith _{&} Nephew was used to study the development of bacterial resistance against Ag. Acticoat® (1 cm² area) was placed in water (1 mL) and kept at room temperature for 24 h in order to promote leaching of Ag ions. ICP-MS analysis of leached solution was done to quantify the amount of Ag ions. The leached Acticoat® solution (Ag⁺,0.06 ug/mL; sub-minimum inhibitory concentration, sub-MIC) was used to incubate with E.coli or S.aureus suspensions (10⁵ CFU/mL) for 20 h in 10% human serum (HS; v/v in PBS). Then, for antimicrobial test, 10⁵ CFU/mL of bacteria from passage 1 were incubated with 0.1 ug/mL (minimum inhibitory concentration, MIC) of Acticoat® solution in 10% human serum (HS) for 6 h followed by serial dilution and plating. Plates were incubated at 37° C. for 24 h. The visible bacteria grown on plates were counted. For next passage, bacterial suspension was added in fresh 10% HS containing 0.06 ug/mL of Acticoat® leached solution for 20 h. Next day, antimicrobial test was done as mentioned above. Same cycle was done for each passage. Similarly, to study the bacterial resistance against LL37-Au NPs, 10 ug/mL and 30 ug/mL of LL37-Au NPs were used as sub-MIC and MIC. Bacterial resistance study was done as discussed above.

Atomic force microscopy (AFM) analyses. PU-Adhesive-LL37NP dressing was dried completely under N₂ gas before acquiring the image. The morphology of NPs on the surface was acquired in non-contact mode as described below. E.coli suspension incubated on PU-Adhesive-LL37NP and PU dressings for 18 h (as discussed above) was washed gently with PBS and then fixed with 2.5% glutaraldehyde. The morphology of bacteria was acquired immediately by AFM. Agilent Technologies 5100 (USA) measurement system has been used in non-contact mode in ambient air environment. The APPNano ACT (USA) non-contact mode silicon cantilevers with typical resonance frequency of 300 kHz have been used.

Cell Culture. Human dermal fibroblast cells (NDHF) (Lonza) were cultured with Dulbecco’s modified eagle’s medium (DMEM) (Sigma) supplemented with 10% (v/v) of fetal bovine serum (Gibco, Grand Island, NY, USA) and 1% (v/v) penicillin/streptomycin (Lonza). NDHF cells under passage number ten were used in all experiments. Human keratinocyte cell line (HaCaT cell line, CLS, Eppelheim, Germany) was cultured as recommended by the vendor. Briefly, HaCaT cells were cultivated using DMEM supplemented with 1% (v/v) penicillin and streptomycin (Invitrogen) and 10% (v/v) fetal bovine serum (FBS, Invitrogen) until 90% of confluence. For passage, HaCat cells were initially trypsinized and then scraped. The cells were sub-cultured at a ratio of 1:3 until achieving the number of cells required for the experiment.

Biocompatibility study. Keratinocytes (2×10⁴ cells) were cultured on top of PU-Adhesive-LL37NP, PU-Adhesive or PU films and cultured for 4 and 24 h. At different time periods, HaCaT cells were trypsinized and cells were collected for CellTiter-Glo® luminescent cell viability assay (Promega) to assess the ATP production in cells according to the supplier’s instructions. To study the cytotoxicity effect of extracts from PU-Adhesive-LL37NP, Acticoat® and PU-Adhesive films to normal human dermal fibroblast (NDHF) cells, 3 cm² of each dressing was incubated in DMEM media for 24 h. Then, the dressings were taken out from media and media was supplemented with 10% FBS. NDHF cells, at a density of 5.000 cells per well in a 96 well plate, were incubated with different dilutions of the extracts for 24 h. Cell viability was measured using ATP assay. All experiments were performed in triplicate (n=3) .

In vivo wound healing. 6-7 week old db/db mice (Charles River) were anesthetized with isoflurane and the hair in dorsal area was shaved using an electric shaver and depilatory cream before the surgery. Round shaped full-thickness excision wounds (6 mm²) were made using the skin biopsy punch on both side of the dorsal side of each mouse. 14 mice were assigned per groups and PU-Adhesive-LL37NP and PU dressings were placed randomly on either side of the wounds and fixed the dressings on the mice using transparent Tegaderm adhesive dressing. On days 0, 3, 6, 9, 12 and 14, the pictures of wounds were digitally photographed with the same optical zoom. Wound areas were quantified using Image J software. Wound sizes at different days of healing were expressed as percentage of the initial respective wounds. At days 6 and 14, mice were sacrificed and wound tissues were collected for Hematoxylin and Eosin (H&E) staining, immunofluorescence, qRT-PCR and ICP-MS analyses.

Histology analysis. After sacrificing mice, the wound halves were immediately fixed with paraformaldehyde (4% in PBS, 0.01 M, pH 7.4) and stored at 4° C. Wound tissue was embedded in paraffin blocks and sequentially sectioned at 5 µm thickness using a MICROM 17M325 microtome (Thermo Fisher Scientific, DE). Skin sections from days 6 and 14 were stained with H&E to assess the different stages of healing. Images were taken with an AxioCam camera on an Axioplan microscope (Carl Zeiss GmbH, Oberkochen, DE). All histological analyses were performed on at least 3 wounds per group per time point and images presented are representative of all replicates.

Immunofluorescence analyses of wound samples. Before immunofluorescence analyses, wound samples were deparaffinized. The samples antigen retrieval was performed using 10 mM sodium citrate buffer (pH 6) containing 0.05% Tween 20. Next, the samples were washed three times in PBS and permeabilized in 0.2% Triton-X 100 for 10 min at room temperature. Following the permeabilization step, the samples were once again washed in PBS, and incubated for 1 h at room temperature with the blocking agent such as 5% (v/v) BSA. Promptly after the blocking stage, the tissue samples were again incubated at 4° C. for overnight in the following primary antibodies: MMR/CD206 (mouse, 1:38; R&D Systems, EUA), CD80 (rabbit, 1:100; Abcam, UK), keratin 14 (rabbit, 1:1000; BioLegend, USA), keratin 5 (chicken, 1:200; BioLegend, USA). After the incubation with the primary antibodies, the tissue samples were washed in PBS and incubated in the following antibodies: Alexa Fluor 488 Donkey Anti-Goat (1:800; ThermoFisher Scientific, USA), Cy™3 - conjugated affiniPure goat anti-rabbit (1:800, Jackson Immuno Research, UK), Alexa 633 goat anti-Rabbit (1:500; Thermo Fisher Scientific, USA) and Alexa 488 goat anti-chicken (1:500; Thermo Fisher Scientific, USA) for 1 h at room temperature in a dark room. The samples were washed again in PBS and incubated in 4′,6′-diamino-2-fenil-indol (2 µg/mL, DAPI, Sigma) for 10 min and re-washed in PBS. The images were acquired using the INCell Analyzer 2000 (ThermoFisher Scientific, USA), in the Cy5, FITC and DAPI channels, and then analyzed using the Image-J Software (National Institutes of Health, USA).

For the quantification of K14 and K5 expressing cells, the ratio of the targeted K14 or K5 expressing cells with the total number of the cells were quantified. The K14 and K5 expressing cells on the wound gap and proliferative areas of the wound (wound edges) was quantified. In addition, the thickness of the proliferative area of the wound and wound gap was measured. The inventors took 15 measurements and calculated the average of the measurements. The inventors also calculated the florescence intensity of the presence of the K14 or K5 expressing cells using the following formula:

$$\text{Ir = Ar} \left({\text{I}}_{\text{Ar}} - {\text{I}}_{\text{background}}\right) *$$

$$\begin{matrix}* \left({\text{Ar = Area of intensity; I}}_{\text{ar}} = {\text{mean of interest; I}}_{\text{background}} =\right) \\ \text{intensity of the} \left(\text{background}\right) .\end{matrix}$$

For the quantification of the pro-inflammatory macrophages (M1 macrophages) and anti-inflammatory macrophages (M2 macrophages), it was done a ratio of all the CD80⁺ or CD206⁺ positive-labeled cells and the total number of cells in wound tissue samples. In addition, it was calculated the percentage of co-localization between M1 macrophages and M2 macrophages, using the JaCoP plugin (Just another Colocalization Plugin) on ImageJ software, in order to observe the transition of M1 subtype to M2 subtype of macrophages in day 6 wound tissues.

Gene analysis (qRT-PCR) of wound samples. The gene expression analysis was performed to estimate the presence of different cytokines in wounds treated with LL37-AuNP-PU and PU dressings. The targeted genes were: tumor Necrosis Factor Alpha (TNFα), interleukin-6 (IL6), and interleukin-10 (IL10). Total RNA extraction of frozen wound tissues was performed using RNeasy® Fibrous Tissue Mini kit (QIAGEN, Germany), accordingly to the manufacturer recommendation, and the quantification of total RNA, as well as, the purification levels were performed using the NanoDrop 2000 Spectrophotometer (ThermoFisher Scientific). Following the RNA extraction, the cDNA synthesis was performed using qScript® cDNA Supermix kit (Quantabio, USA), also according to the manufacturer’s recommendation. The samples were amplified for 5 minutes at 25° C., 30 minutes at 42° C., 5 minutes at 85° C. and held at 4° C. in the CFX Connect Real-Time PCR Detection System (BioRad, USA). Lastly, the qRT-PCR protocol used was the NZYSpeedy qPCR Green Master Mix (2x), ROX (nzytech, Portugal). qRT-PCR analysis was run for 40 cycles in the CFX Connect Real-Time PCR Detection System (BioRad, USA). The minimal cycle threshold (Ct) values were automatically calculated using the BioRad CFX Maestro software and the quantification of the targeted genes were normalized to the GAPDH gene using the Livak Method (Fold difference in the expression= 2^{-ΔΔCt} ). Primers of target genes and reference gene used for qRT-PCR analyses, designed by Sigma, are listed below:

TABLE 1

Primers of target genes and reference gene used for qRT-PCR analyses are listed below:

| | | |
|---|---|---|
| | Forward sequence | Reverse sequence |
| GAD PH | AGCCACATCGCTCAGACACC (SEQ ID NO: 2) | GTACTCAGCGCCAGCATCG (SEQ ID NO: 3) |
| TNF-α (SEQ ID NO: 3) | GTCTCAGCCTCTTCTATT (SEQ ID NO: 4) | CCATTTGGGAACTTCTCATC (SEQ ID NO: 5) |
| IL-6 (SEQ ID NO: 4) | ACCTGTCTATACCACTTCAC (SEQ ID NO: 6) | GGCAAATTTCCTGATTATATCCA (SEQ ID NO: 7) |
| IL-10(SEQ ID NO: 5) | CACAAAGCAGCCTTGCAGAA (SEQ ID NO: 8) | AGAGCAGGCAGCATAGCAGTG (SEQ ID NO: 9) |

Statistical analyses. One-way ANOVA statistical analyses and unpaired t-test were performed using the GraphPad Prism 6.0 Software.

### Results

Preparation and characterization of PU-adhesive-LL37NP films. LL37NPs were synthesized as reported previously by the inventors[10]. UV-vis spectrum showed a surface plasmon resonance (SPR) band centered at 530 nm, which agrees with the transmission electron microscopy (TEM) results showing NPs with a spherical geometry with an average diameter of 22 ± 8 nm (n=100) (FIG. 10). To coat LL37NPs on the PU film, a thin layer of UV light sensitive polymer adhesive (111-MSK) was uniformly coated on PU film (1.5 cm²) followed by addition of LL37-Au NPs resuspended in ethanol (60 µg/cm²) and exposure to UV light for 4 min (FIG. 1) . AFM analyses indicated a uniform coating of LL37NPs on PU films (FIG. 2A) although some aggregated structures were also observed due to the drying effect during the coating process. The amount of LL37NPs coated on PU film was quantified by ICP-MS analyses. ICP-MS analyses indicated that approximately 63 µg of LL37-Au NPs was present per cm² on PU-adhesive-LL37NP dressings. Then, stability studies were performed to evaluate the leaching of LL37NPs from PU-adhesive-LL37NP dressings. ICP-MS analyses of the leached solution indicated that only a small amount of LL37NPs (0.4 µg out of 63 µg) was leached from PU-adhesive-LL37NP dressing after 5 days of incubation in PBS solution, indicating that NPs were strongly immobilized on PU film (FIG. 2B). FTIR analyses confirmed the signature of amide-I band, indicating the presence of LL37 peptide on PU-adhesive-LL37NP film while no such band was observed in PU film or polymer adhesive coated PU film (named PU-adhesive from now on) (FIG. 2C). Similarly, XPS analyses showed an increase of atomic % compositions of N, C and S in LL37NPs coated PU film in comparison to PU and PU-adhesive films (Table 2). The presence of S atom in PU-adhesive-LL37NP film was due to LL37 peptide containing thiol (-SH) functional group at C-terminus. Additionally, the signature of Au was also found in PU-adhesive-LL37NP dressing, demonstrating the presence of LL37NPs in the dressing. These results are in line with the contact angle measurements, showing LL37NPs coated PU film had a lower contact angle, due to the presence of LL37NPs, than PU or PU-adhesive films (FIG. 2D and FIG. 10D). Interestingly, PU-adhesive-LL37NP film had a negative zeta potential (-11.3 ± 1.2) while the LL37NP suspension had a positive zeta potential (+16 ± 2 mV) (FIG. 2E). This is explained by the fact that PU-adhesive film had a negative zeta potential result (-13 mV), which became less negative (-4.65 ± 0.51 mV) after the immobilization of LL37NPs (FIG. 2E). Importantly, coating strategy of the present application facilitated higher amount of LL37 peptide (the inventors verified that 30% mass of LL37NPs was LL37, therefore if there is 60 µg NPs/cm² of film, this corresponds to 20 µg peptide/cm²) available on the PU film than previously reported approaches (0.66 and 0.19 µg/cm²) .

TABLE 2

Atomic composition (At%) of different dressings measured using XPS.

| | C1s | N1s | O1s | S2p | Au4f |
|---|---|---|---|---|---|
| PU | 81.39 | 1.54 | 16.99 | 0.08 | 0 |
| PU-adhesive | 94.92 | 2.31 | 4.95 | 0.13 | 0 |
| PU-adhesive-LL37NPs | 68.47 | 5.97 | 24.15 | 1.26 | 0.16 |

In vitro antimicrobial activity of PU-adhesive-LL37NP films. To test the antimicrobial activity of PU-adhesive-LL37NPs, gram-negative (E.coli, P.aeruginosa) and gram-positive (S.aureus) bacteria (10⁵ CFU per cm² dressings; suspended in PBS or 10% HS) suspensions were applied to the surface of the dressings, kept in a humidified chamber in order to prevent the evaporation of the solvent, and then counted after 24 h (FIG. 3A). PU-adhesive and PU films have been used as controls. As expected, PU-adhesive and PU films did not show antimicrobial activity against bacteria suspended either in PBS or 10% HS (FIG. 3B). PU-adhesive-LL37NPs containing 20 µg of LL37NPs (equivalent to 6.6 µg of conjugated LL37 peptide) per cm² showed no antimicrobial activity against E.coli, S.aureus and P.aeruginosa bacteria suspended in PBS (FIG. 11A). In contrast, PU-adhesive-LL37NPs containing 63 µg of LL37NPs per cm² showed antimicrobial activity against the same bacteria (3 log reduction). Importantly, the antimicrobial activity observed was not due to leaching of NPs because the concentration of leached NPs was a very low (FIG. 11B). The amount of LL37 peptide present on PU-adhesive-LL37NP dressing (63 µg of LL37-Au NPs/cm²) was approximately 20 µg/cm², which was considered to be the minimum inhibitory concentration (MIC) of the immobilized LL37. The MIC of the free LL37 peptide against E.coli, S.aureus and P.aeruginosa was up to 15 µg/mL.

Several studies have shown that the antimicrobial activity of immobilized AMPs decreases in the presence of serum most likely due to the formation of protein corona and the degradation of the immobilized peptides[19-21]. To test the effect of HS in the antimicrobial activity of PU-adhesive-LL37NPs, E.coli, S.aureus and P.aeruginosa suspended in 10% HS was incubated with different dressings as mentioned above. It was observed that PU-adhesive-LL37NPs had relatively high antimicrobial activity against E.coli, S.aureus and P.aeruginosa, indicating that HS did not significantly affected the bactericidal activity of PU-adhesive-LL37NP dressing (FIG. 3C).

To investigate the mechanism of antimicrobial activity of PU-adhesive-LL37NP film, AFM analyses of E.coli seeded on top of the film were performed. Height mode AFM images clearly showed that E.coli exposed to soluble LL37 peptides or adhered to PU-adhesive-LL37NPs showed a damaged cell membrane (FIG. 4). The results disclosed herein indicate the formation of bulged-like structures on the cell membrane (indicated by arrows), which could be formed likely due to the replacement of cationic divalent ions from the bacterial outer membrane, leading to penetration of LL37 peptides, which caused cytoplasmic materials to fill the periplasmic space (FIGS. 3B and 3D). In contrast, E.coli in suspension or adhered to PU-adhesive films remained intact (FIGS. 3A and 3C).

Next, the inventors evaluated whether LL37NPs induced bacterial resistance (FIG. 5). For this purpose, E.coli and S.aureus suspensions were serially passaged 16 times in the presence of sub-MIC concentrations (10 µg/mL) of LL37NP suspensions (FIG. 5A). The antimicrobial activity of LL37NPs was then tested against E.coli and S.aureus using MIC of LL37NPs (30 µg/mL) in 10% HS. The results indicated that LL37NPs did not induce resistance in bacteria after exposure for 16 passages at sub-MIC of LL37NPs; however, the antibiotic chloramphenicol was able to induce resistance within 3 days (FIG. 5B). Importantly, the inventors also performed bacterial resistance assay using silver ions leached from Acticoat® wound dressing. Acticoat® dressing was incubated in PBS for 1, 3 and 5 days followed by the quantification of silver ions using ICP-MS analysis (FIG. 11C). There was progressive increase in the amount of Ag ions in solution with increasing incubation time (FIG. 11C). Antimicrobial test was performed using leached silver ion solution against E.coli and S.aureus. It was found that MICs of the leached silver ion solution were 0.1 and 0.3 µg/mL for E.coli and S.aureus respectively (FIG. 5C). Bacterial resistance assay showed that both bacteria developed a small resistance after 5 passages of sub-MIC of silver ion leached solution. After 10^{th} cycles, when concentration of silver ion leached solution was increased 2 times to MICs, the complete reduction of bacterial population was observed until 16^{th} cycle. Overall the results showed that PU-adhesive-LL37NP dressings had a potent antimicrobial property against both gram-positive and gram-negative bacteria and did not induce bacterial resistance after 16 cycles of exposure to bacteria.

In vitro cytotoxicity of PU-adhesive-LL37NP films. Human keratinocytes were chosen as a representative cell type in the skin with which the dressing may interact with. To evaluate the cytotoxicity of films, keratinocytes were cultured on the top of PU-adhesive-LL37NP, PU-adhesive and PU films for 4 and 24 h and then cell viability was evaluated by an ATP assay (FIG. 12A). As controls, keratinocytes cultured on tissue culture poly(styrene) (TCPS) and in the presence of soluble LL37 peptides (similar concentration to the immobilized peptide in the films) were used. No statistic difference in keratinocyte metabolism was observed after incubation with the different films or soluble peptide for 4 and 24 h. These results indicate that PU-adhesive-LL37NP film was not cytotoxic against keratinocytes. In addition, the extracts from PU-adhesive-LL37NP, PU-adhesive and PU films had little effect on metabolism of fibroblasts. For comparison, the inventors have used the extract of Acticoat® (same area), a silver-based dressing. In this case, the extract of Acticoat® (corresponding to a concentration of silver of 0.2 µg/mL) had pronounced effect on fibroblast metabolism (FIG. 12B). Altogether, these results indicated that PU-adhesive-LL37NP films were non-cytotoxic.

In vivo evaluation of wound healing potential of PU-adhesive-LL37NP dressing. The wound healing potential of PU-adhesive-LL37NP in diabetic type II mice (db/db genetic model) was evaluated. Wounds made on the dorsal side of mice were treated with PU-adhesive-LL37NP or PU (control) dressings for 14 days (FIG. 6A). The progress of wound healing was monitored regularly by measuring the wound area (FIG. 6B). PU-adhesive-LL37NP dressing accelerated wound healing as compared to PU dressing (FIG. 6B and C). On days 6 and 9, wounds treated with PU dressing showed 5% and 38% healing whereas the ones treated with PU-adhesive-LL37NP dressing showed 10% and 60% healing, respectively. On day 14, more than 90% of wound area was healed after the treatment with PU-adhesive-LL37NPs while 75% of wound area was healed with PU dressings. H&E staining showed a low granulation tissue formation in wounds at day 6 in both conditions; however, higher granulation tissue at day 14 in wounds treated with PU-adhesive-LL37NP relatively to the ones treated with PU dressing (FIG. 6F). Indeed, at day 14, a prominent thick and dense epithelial layer was formed on the wounds treated with PU-adhesive-LL37NP dressing, while a thin epithelial layer was observed in the wounds treated by the PU dressing. Additionally, H&E staining images show tissue remodeling in wounds treated with both dressings at days 14 (FIG. 6F).

The enhanced wound healing activity of PU-adhesive-LL37NP dressing could be due to the leaching of LL37NPs from the dressing, since LL37NPs have wound healing properties when internalized by skin cells [12] . Therefore, the inventors quantified the amount of LL37NPs leached from the PU-adhesive-LL37NP dressings to the wound bed, skin surrounding the wound and liver using ICP-MS analyses. In addition, the inventors quantified by ICP-MS the LL37NPs that remained in the dressing after animal testing at days 6 and 14 days. A very small amount of LL37NPs (less than 1.5 µg from 63 µg coated on PU-adhesive-LL37NP dressing) was present in the wound bed, skin surrounding the wounds and liver at days 6 and 14 (FIG. 6D). In addition, less than 9 µg LL37NPs leached from PU-adhesive-LL37NP dressings after 14 days in contact with wounds (FIG. 6E).

In chronic wounds, the dressings are often changed to allow medical staff to remove dead or inflamed tissue (known as debridement)[2]. Therefore, it was hypothesized whether the healing properties of the PU-adhesive-LL37NP dressings would remain if they were removed at day 6 (when the beginning of the bioactivity is noticed). Thus, wounds made on the dorsal side of mice were treated with PU-adhesive-LL37NP or PU (control) dressings for 6 days, after which they were removed and the healing process monitored until day 14 (FIG. 7). Interestingly, wounds treated with PU-adhesive-LL37NP dressings maintained an accelerated healing relatively to wounds treated with PU dressings, showing that 6 days (or even less) of contact is necessary to improve the wound healing response. As in the previous animal experiments, the presence of LL37NPs in the wound bed was relatively low (below 0.3 µg per wound) (FIG. 7D). H&E staining analyses showed the wound re-epithelization was completed and a thick scab of epithelial layer was formed on the wound treated with PU-adhesive-LL37NP dressings; however, such tissue remodeling was not observed in the wound treated with PU dressing (FIG. 15E). Overall, the results disclosed herein indicate that PU-adhesive-LL37NP dressings accelerate wound healing in a diabetic type II animal model. In addition, the results seem to indicate that most of the bioactivity of the dressing was mainly mediated by tissue contact and not by the leaching of LL37NPs in the wound bed.

In vivo regenerative mechanism of PU-adhesive-LL37NP dressings. Previous studies have shown that LL37 significantly improved re-epithelization and granulation tissue formation in healing impaired ob/ob mouse model[25], re-epithelization and vascularization in dexamethasone-treated mouse[26] or in acute wound healing animal mouse[27]. Yet, it is unknown the regenerative mechanism of LL37 immobilized to a substrate preventing its cellular uptake, and thus being its effect mediated mostly by tissue contact. Therefore, the inventors studied the wound healing mechanism of PU-adhesive-LL37NP dressings focusing in the re-epithelization and immunomodulatory properties of the dressing. The inventors performed immunofluorescence analyses of day 6 wounds to evaluate the expression of keratin 14/5 (K14/5) (FIG. 8). K14/5 are highly expressed in basal layer of epidermis and thus required for normal development and functioning of basal cells[28]. Their expression is down regulated and gradually reduced as these cells moved upward and differentiate in wounds. Wounds treated with PU-adhesive-LL37NP dressings showed higher expression of K14 (both in intensity as well as width) than wounds treated with PU dressing (FIG. 8A). In addition, the length of proliferative edges (as evaluated by the expression of K14 and K5) was more significant in wounds treated with PU-adhesive-LL37NPs than wounds treated with PU dressings. Moreover, wounds treated with PU-adhesive-LL37NPs showed a statistical significant decrease in wound gap as compared to PU dressings (FIG. 8 and FIG. 13). Macrophages have a critical role in skin wound healing[29]. During the progression of normal wound healing process, there is a transition of macrophage cell phenotype from pro-inflammatory (M1) in early stage to anti-inflammatory (M2) in late stage to coordinate the regeneration of skin[30]. Importantly, macrophages hyperpolarize towards both M1 and M2 in early wound healing in normal condition while continue to express M1 phenotype in late stage of healing in diabetic wound condition[31, 32]. Immunofluorescence analysis of day 6 wounds shows the expression of both M1 (CD80⁺) and M2 (CD206⁺) phenotypes of macrophage cells in wounds treated with both dressings. Interestingly, the percentage of macrophages polarized for either M1 or M2 phenotypes was higher in wounds treated with PU-adhesive-LL37NP dressings than with PU dressings (FIG. 9A). It is interesting also to note that the percentage of M1 or M2 macrophages in wounds treated with both dressings was relatively similar.

The presence of double positive CD80/CD206 cells indicates the switching of macrophage phenotype at day 6 wounds (FIG. 8B and D). Although the mixed population of M1 and M2 phenotypes is found in wounds, PU-adhesive-LL37NP dressings stimulate higher level of M1 to M2 phenotype switching as compared to PU dressings (FIG. 9). Interestingly, the opposition trend is observed in day 14 wound treated with PU-adhesive-LL37NP dressings in which the population of M1 macrophages decreases while the population of M2 macrophages increases with healing time, indicating the presence of anti-inflammatory environment in the wounds (FIG. 9C). Curiously, the inventors observed that the number of M1 macrophages in day 14 wounds is higher than the day 6 wound treated with PU dressings (FIG. 9C). At same time, PU dressings promote the presence of more M2 than M1 macrophages in day 14 wounds. To determine the anti-inflammatory property of PU-adhesive-LL37NP dressings, a quantitative evaluation of the expression of different cytokines such as TNF-α, IL6 and IL10 was performed at mRNA level (FIG. 9E). FIG. 9E shows the levels of pro-inflammatory cytokines such as TNF-α and IL6 are higher in day 6 wounds with prolonged inflammation followed by their significant decrease in day 14 wounds after treatment with PU-adhesive-LL37NP dressings. Such drastic effect was not observed in wounds treated with PU dressings. In contrast, PU-adhesive-LL37NP dressings promote higher expression of anti-inflammatory IL10 cytokine in wounds with the progression of healing of wounds as compared to PU dressings. The expression of pro and anti-inflammatory cytokines in wounds are in well agreement with immunofluorescence analysis where the transition of M1 to M2 macrophages are observed from day 6 to 14 after treatment with PU-adhesive-LL37NP dressings.

### DISCUSSION

It is herein disclosed a process to produce an antimicrobial peptide-coated film that presents simultaneously antimicrobial and skin healing properties. The film has bactericidal activity against gram-negative and gram-positive bacteria and low propensity to induce bacteria resistance after multiple exposures. Moreover, when applied in diabetic wounds enhance skin re-epithelization mediated by an increase in keratin-14 positive cells at the proliferative wound edges and by an increase in macrophages M1 and M2 in the wound bed at day 6. The wound dressing proposed here activates the healing response mainly by contact with skin tissue and not by the leaching of its components.

In this work, the inventors have selected LL37 peptide to immobilize in the dressing because it is an endogenous AMP and a master regulator of skin homeostasis. This peptide is downregulated in the epidermis of diabetic foot ulcers and chronic venous ulcers and thus the presentation of this peptide in the wound bed may be beneficial to promote wound healing. Therefore, the inventors have coated LL37NPs (≈ 60 µg/cm²) on PU films using a UV responsive polymer adhesive. The antimicrobial peptide-coated film had relatively high bactericidal activity against gram-positive and gram-negative bacteria even in the presence of human serum. Importantly, LL37NPs did not stimulate bacterial resistance after repeating exposure for 16 cycles however; the leached Ag ions from commercially available Acticoat® dressing induce the resistance in gram-positive and gram-negative bacteria within 10 days.

So far it is relatively unknown whether immobilized LL37 immobilized in a film and thus not taken up by skin cells would be effective in promoting wound healing. In the present work, the LL37-containing film was in contact with the borders of the wound while the middle of the film was likely in contact with blood and immune cells. Therefore, the inventors decided to investigate re-epithelization and immunomodulatory processes mediated by the PU-adhesive-LL37NP dressings. Importantly, the inventors did not observe significant leaching of LL37NPs from the dressings in skin wounds, showing that most of the bioactivity of the dressing was mediated by tissue/cell contact.

LL37 peptides play an important immunomodulatory role in skin wounds [34]. It has been shown that LL37 peptides reduce the production of TNF-α from M1 and M2 macrophages but make M2 phenotype more anti-inflammatory to produce IL-10 [30]. The prolonged presence of M1 macrophage in wounds leads to continued inflammation and the foreign body reaction and therefore the switching of M1 to M2 phenotypes in the later stage are crucial for effective wound healing. Although switching of M1 to M2 phenotype in diabetic wounds generally delayed [35], it is showed herein that, PU-adhesive-LL37NP dressings initiated the higher polarization of M2 from M1 phenotype in diabetic wounds at day 6 as compared to PU dressings where such trend was not observed. The analysis of CD80 and CD206 markers indicates the presence of mixed macrophage population at day 6 of wound healing rather than single polarization. Additionally quantitative-PCR analysis also indicates the expression of more anti-inflammatory IL10 cytokine in wounds treated with PU-adhesive-LL37NP dressings for 14 days. Importantly, PU adhesive-LL37NPs dressings stimulate higher level of polarization of M1 to M2 phenotypes at day 6. Additionally at same time, PU adhesive-LL37NPs dressing promotes the higher production of K14/5 in wounds at day 6 in order to facilitate the formation of stratified epithelial layer in diabetic wounds at the later stage of healing process.

An important clinical criterion for the development of effective wound dressing is their single application to heal wounds because it minimizes the frequent visit of patients to hospital to change the dressings and therefore reduces the discomfort and the pain along with reduction in the healthcare expenses. The cost to prepare 1 cm² PU adhesive-LL37NPs dressing is approximately 0.5 euro, which is 2 times cheaper than commercially available Acticoat® dressing. In-vivo data show that 1 cm² PU adhesive-LL37NPs dressing heals efficiently 6 mm diameter diabetic wound in a single application. Importantly, PU adhesive-LL37NPs dressings accelerate wound-healing process by promoting re-epithelialization, keratinization and induction of immune cells in wounds.

A novel and bioactive LL37-Au NPs coated PU dressing was successfully fabricated to promote rapid wound healing. Taking advantage of the conjugated LL37, PU-adhesive-LL37NP dressings have potent antimicrobial activity against Gram-positive and negative bacteria in the human serum but do not induce antimicrobial resistance in bacteria as compared to commercially available Acticoat® dressing. This study demonstrates that PU-adhesive-LL37NP dressings chemotaxis macrophage cells in wounds and help them to switch from M1 to M2 phenotypes at days 6 in diabetic conditions. PU-adhesive-LL37NP dressings also induce the expression of Keratin 14/5 in proliferative edge of wounds, promoting the rapid closure of wound gap. Importantly, in vivo wound healing evaluation in diabetic mice indicates that PU-adhesive-LL37NP dressings accelerate faster wound healing compared with PU dressings. The negligible amount of LL37-Au NPs leached in wounds was observed in wounds and liver, indicating that PU-adhesive-LL37NP dressings are biocompatible. Finally, the prepared PU-adhesive-LL37NP dressings have potential applications in treating burns, chronic and diabetic wounds along with the prevention of bacterial infection.

Several features are described hereafter that can each be used independently of one another or with any combination of the other features. However, any individual feature might not address any of the problems discussed above or might only address one of the problems discussed above. Some of the problems discussed above might not be fully addressed by any of the features described herein. Although headings are provided, information related to a particular heading, but not found in the section having that heading, may also be found elsewhere in the specification.

### BRIEF DESCRIPTION OF DRAWINGS

For easier understanding of this application, figures are attached in the annex that represent the preferred forms of implementation which nevertheless are not intended to limit the technique disclosed herein.

FIG. 1 illustrates a schematic diagram represents coating of LLNPs on PU dressing and their antimicrobial and wound healing properties.

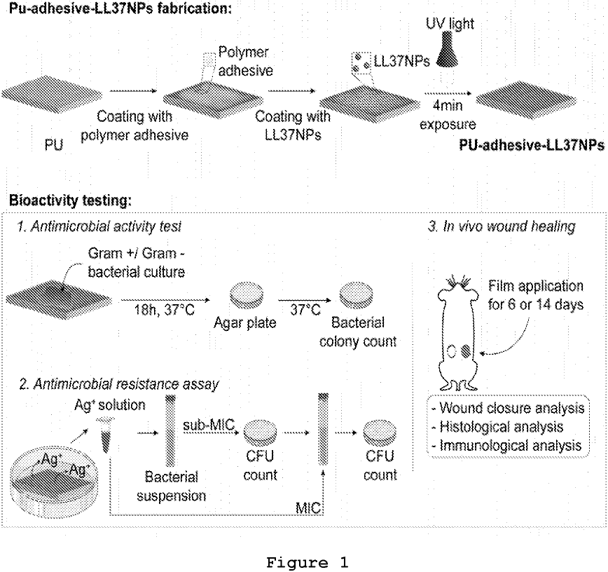

FIG. 2 shows dressing physicochemical characterization. (A) AFM image of PU-adhesive-LL37NPs dressing. (B) ICP-MS analyses of leached LL37NPs from PU-adhesive-LL37NPs dressings incubated in PBS. FTIR spectra (C), contact angle (D) and zeta potential (E) measurements of PU, PU-adhesive and PU-adhesive-LL37NPs dressings.

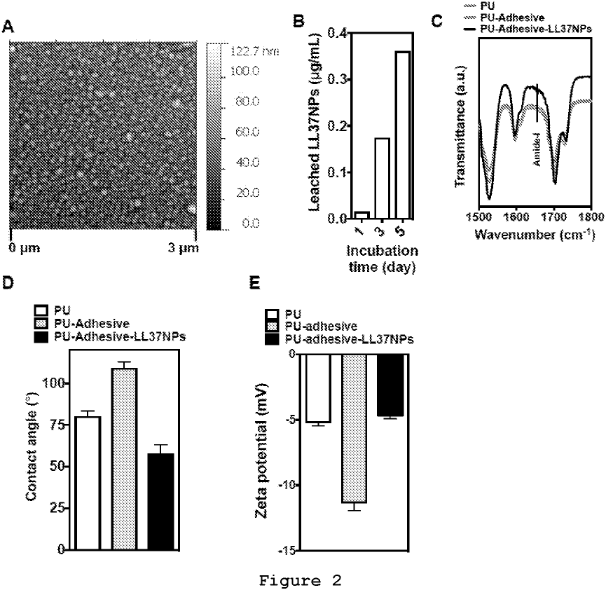

FIG. 3 shows Antimicrobial testing. (A) Schematic representation of the antimicrobial test. Approximately 500.000 bacteria suspended in 100 µL of media (TSY or 10% human serum) were placed in a dressing (1 cm²) for 20 h and then plated in TSY agar for 24 h before counting the colonies. (B, C) Antimicrobial activity of different dressings against E.coli, P.aeruginosa and S.aureus incubated in PBS (pH 7.2) (B) or in 10% human serum (C). In B and C, results are average ± SD, n=5. Statistical analyses were performed by One-way ANOVA followed by a Tukey’s post-test, ****P < 0.0001.

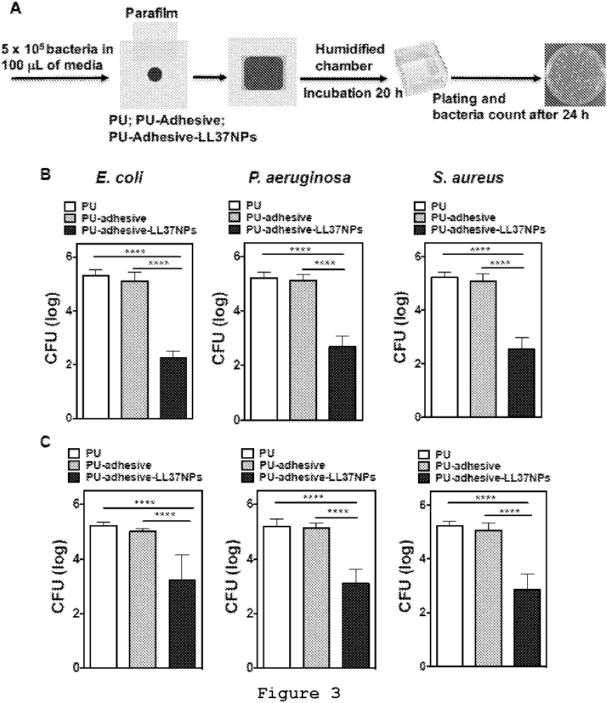

FIG. 4 shows Bacteria morphology after contact with soluble or immobilized LL37 peptide. Height mode AFM images of E.coli: (A1) in suspension, (B1) in suspension in the presence of LL37 peptide (20 µg/mL) (C1) adhered to PU-adhesive films and (D1) adhered to PU-adhesive-LL37NPs films. Figures from A2 to D2 show line profile images of corresponding height mode images.

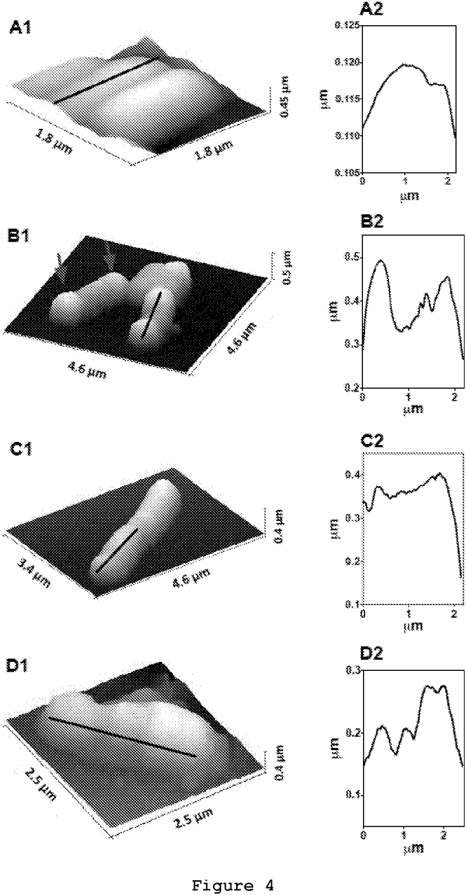

FIG. 5 shows Bacteria resistance testing. (A) Schematic diagram showing experimental procedures being used for bacterial resistance assay. (B) Results of resistance assay with LL37NPs and chloramphenicol with E.coli and S.aureus. (C) Antimicrobial activity of leached silver from Acticoat dressings against E.coli and S.aureus. Acticoat dressing (1 cm²) was incubated in PBS (1 mL, pH 7.2) for 1 day to collect the leached product. ICP-MS analysis was performed to quantify the amount of Ag. (D) Result of resistance assay with the leached silver against E.coli and S.aureus. After 10^{th} cycle, a higher concentration of leached silver was used to show that resistant bacteria may be killed by higher concentration of silver. In B, C and D, results are average ± SD, n=3.

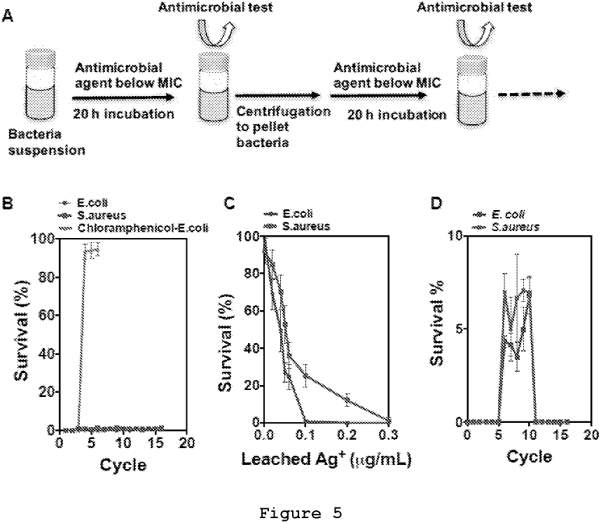

FIG. 6 shows In vivo wound healing properties of PU-adhesive-LL37NPs. (A) Schematic representation of wound healing experiments performed in diabetic mice (db/db mice) using PU-adhesive-LL37NPs and PU dressings. (B) Images of wounds treated with PU or PU-adhesive-LL37NPs taken at different times during the healing process. (C) Quantification of wound area measured from the optical images. Results are average ± SEM (n=7 animals). Statistical analyses were performed by One-way ANOVA followed by a Tukey’s post-test, *P<0.01. (D) ICP-MS analysis of NPs present in wounds, skin around wounds and liver, which are leached from PU-adhesive-LL37NP dressings. Results are average ± SEM (n=6 animals). (E) ICP-MS analysis of LL37NPs present in PU-adhesive-LL37NP dressings applied on wounds for 6 and 14 days. Results are average ± SEM (n=6 animals). Statistical analyses were performed by One-way ANOVA followed by a Tukey’s post-test, *P<0.05 (F) H&E images of wounds at days 6 and 14 treated with PU-adhesive-LL37NPs and PU dressings. Scar bars in all images correspond to 100 µm.

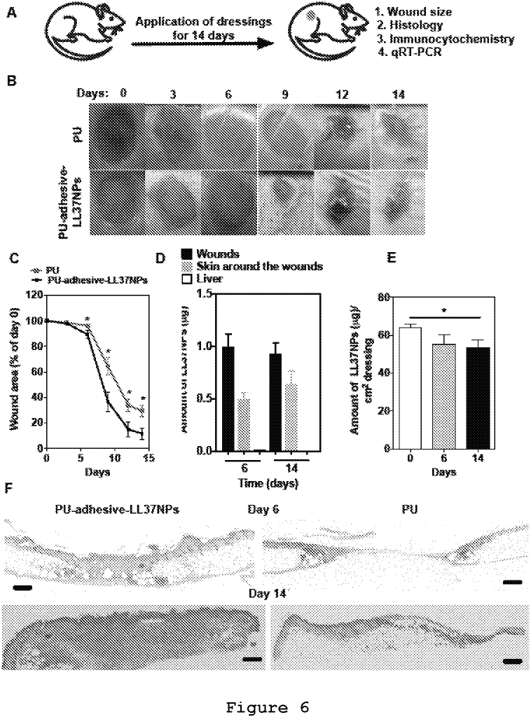

FIG. 7 shows In vivo wound healing properties of PU-adhesive-LL37NPs. (A) Schematic representation of wound healing experiments performed in diabetic mice using PU-adhesive-LL37NP and PU dressings. (B) Optical images of wounds taken at different times of healing process. (C) Quantification of wounds area measured from the optical images (n=7 animals; 2 wounds per animal). (D) ICP-MS analyses of LL37NPs present in wounds leached from PU-adhesive-LL37NP dressings. Results are average ± SEM (n=6 animals; 2 wounds per animal). Statistical analyses were performed by One-way ANOVA followed by a Tukey’s post-test, *P<0.01, **P<0.001. (E) H&E stained images of wounds treated with PU-adhesive-LL37NP and PU dressings. Scale bar corresponds to 100 µm.

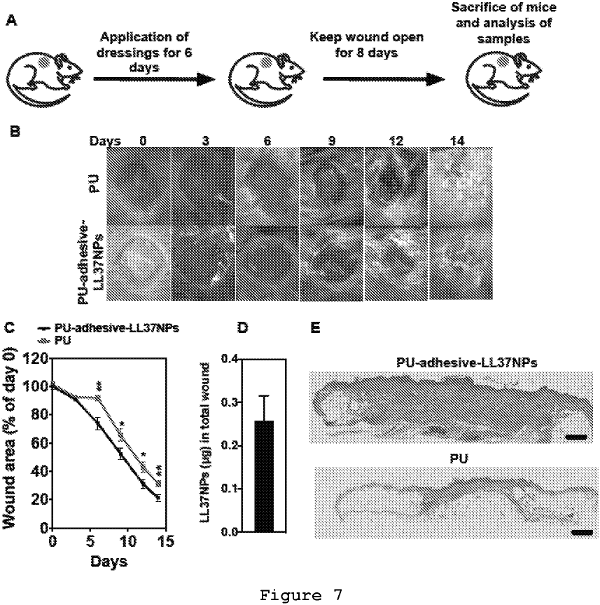

FIG. 8 shows In vivo wound healing mechanism mediated by PU-adhesive-LL37NP dressings: re-epithelization. (A, B) Immunofluorescence analyses of wounds at days 6 to show expression of keratin 14 and 5 after treatment with PU-adhesive-LL37NPs (A) and PU (B) dressings. (C) Quantification of fluorescence intensity, thickness of keratin 14 in wound slides and proliferative length as well as wound gaps at day 6. Results are average ± SEM (n=6 animals). Statistical analyses were performed by unpaired t-test, ****P<0.0001, **P<0.0016.

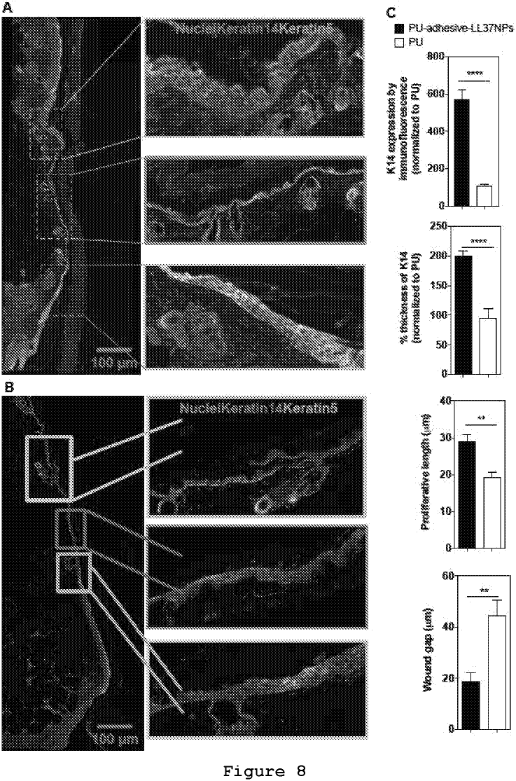

FIG. 9 shows In vivo wound healing mechanism mediated by PU-adhesive-LL37NP dressings: immunomodulation. (A, B) Quantification of M1 and M2 phenotype macrophage cells in wounds at days 6 (A) and 14 (B) treated with PU-adhesive-LL37NPs and PU dressings. (C, D) Immunofluorescence analyses of co-localization of M1 and M2 phenotype macrophage cells at day 6 in wounds treated with PU-adhesive-LL37NPs (D.1) or PU (D.2) dressings. Arrows show co-localization of M1 and M2 phenotypes in different cells. Results are average ± SEM (n=6 animals). (E) qRT-PCR analysis of TNF-α, IL6 and IL19 cytokines in wounds treated with PU-adhesive-LL37NPs and PU dressings. Results are average ± SEM (n=6). Statistical analyses were performed by One-way ANOVA followed by a Tukey’s post-test, *P<0.01, **P<0.001.

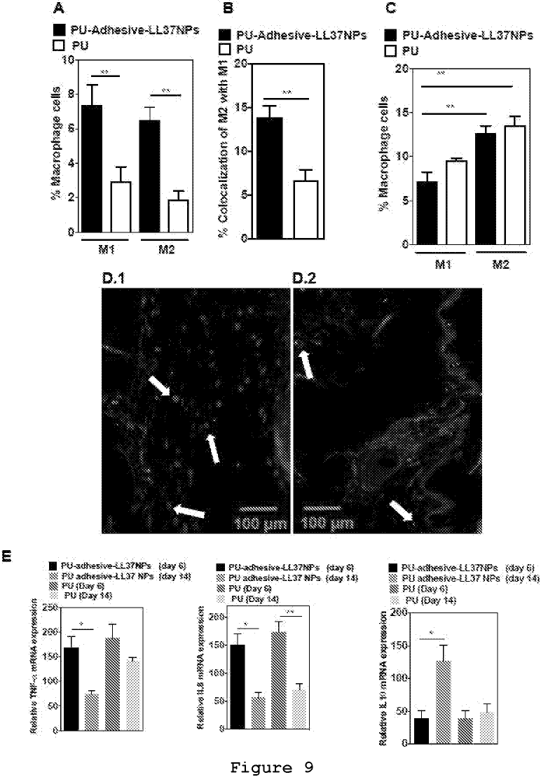

FIG. 10 shows Physicochemical characterization of LL37NPs and films coated with LL37NPs. (A) UV-vis spectrum of LL37NPs. (B) Representative TEM image of LL37NPs. (C) Quantification of particle size of LL37NPs (n=100) from TEM images. (D) Water contact angle images of PU, PUadhesive and PU-adhesive-LL37NP films (n=4, average ± SD).

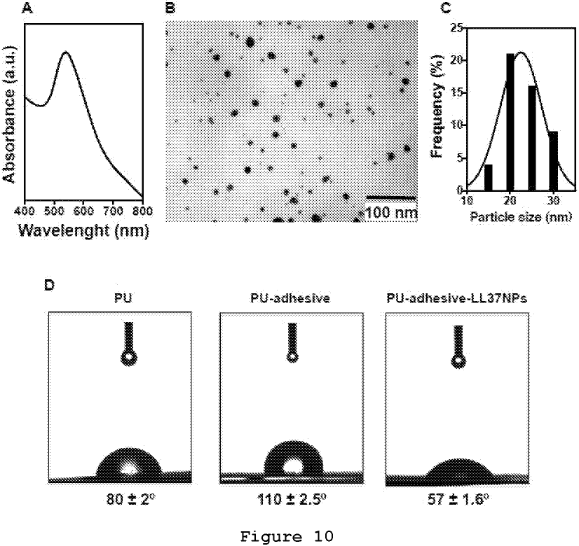

FIG. 11 shows Antimicrobial activity of PU-adhesive-LL37NP films. (A) Antimicrobial activity of PU, polymer adhesive-PU and CureMat (20 µg/cm2) against E.coli, P.aeruginosa and S.aureus in PBS. (B) Antimicrobial activity of leached solution from CureMat and PU dressings (C) ICP-MS analysis of leached silver (Ag) and gold (Au) from Acticoat and CureMat dressings respectively incubated in PBS for different time.

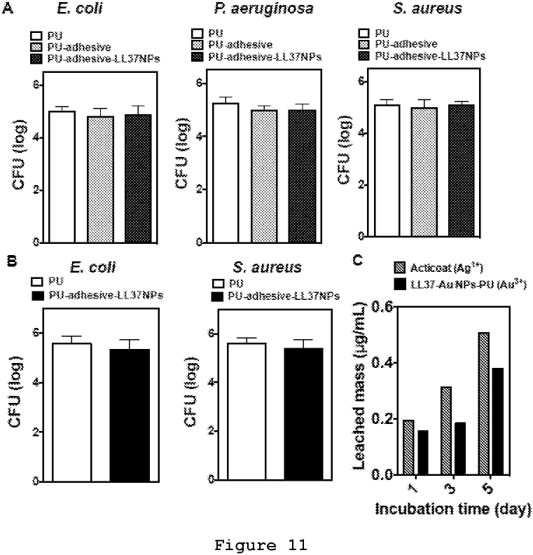

FIG. 12 shows Cytotoxicity of PU-adhesive-LL37NPs against skin cells. (A) ATP production in keratinocytes seeded on top of PU, PU-adhesive or PU-adhesive-LL37NP films for 4 or 24 h. As controls, cells were cultured in tissue culture poly(styrene) (TCPS) with and without LL37 peptide (20 µg/mL). Results are average ± SEM (n=3). (B) ATP production in fibroblasts cultured in TCPS and exposed to extracts of PU-adhesive-LL37NPs and Acticoat dressings. Control represent fibroblast cells grown on TCPS. The area of acticoat was similar to PU-adhesive-LL37NPs and both dressings were incubated in PBS (pH 7.2) at room temperature for 24 h. Results are average ± SEM (n=8).

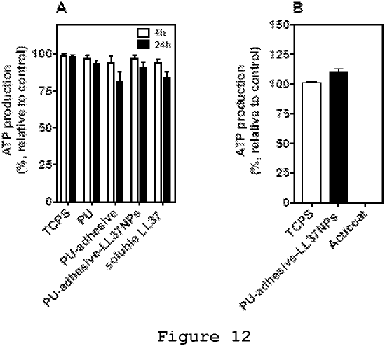

FIG. 13 shows quantification of fluorescence intensity, thickness of keratin 5 (K5) in wound sides, proliferative length and wound gaps of day 6 wounds. Results are average ± SEM (n=6). Statistical analyses were performed by unpaired t-test, ****P<0.0001, **P<0.0094, *P<0.0096.

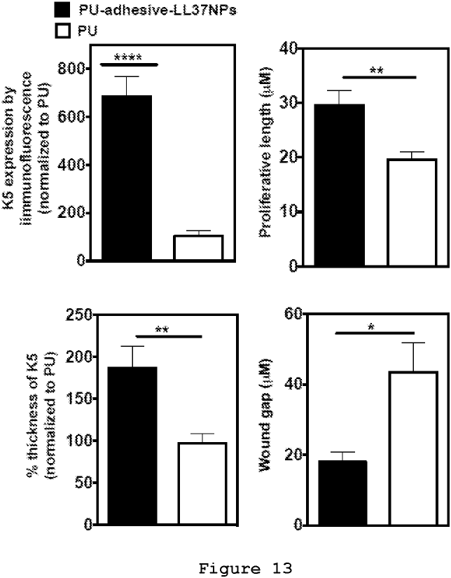

FIG. 14 shows immunofluorescence analysis of M1 (A) M2 (B) phenotype macrophage cells in wound treated with PU-adhesive-LL37NPs for 6 days.

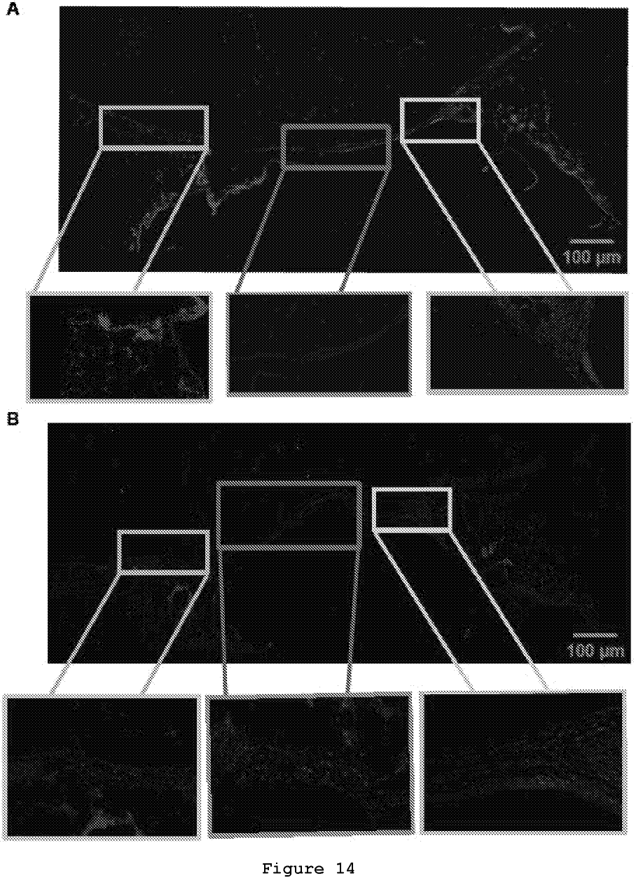

FIG. 15 shows immunofluorescence analysis of M1 (A) M2 (B) phenotype macrophage cells in wound treated with PU dressings for 6 day.

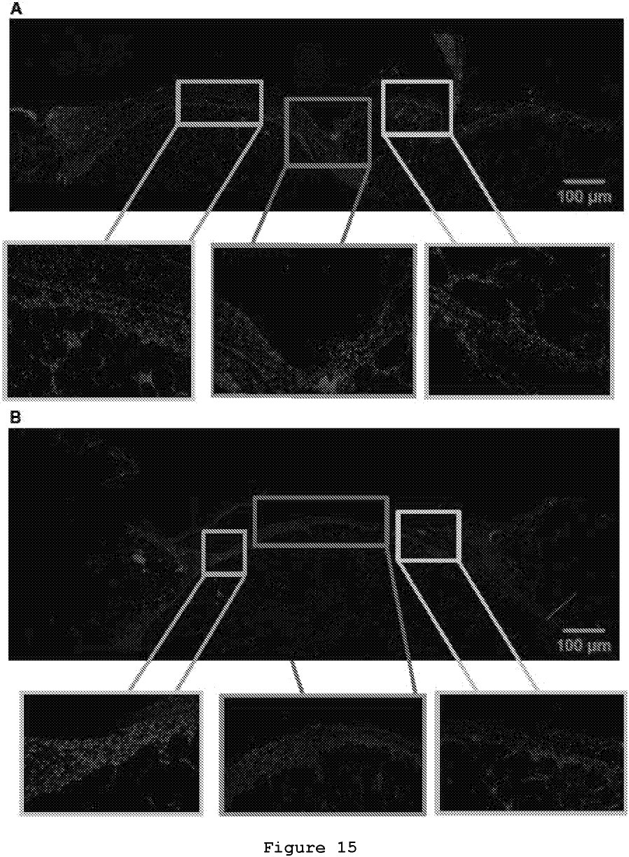

### DESCRIPTION OF EMBODIMENTS

Now, preferred embodiments of the present application will be described in detail with reference to the annexed drawings. However, they are not intended to limit the scope of this application.

In the context of the present patent application, “medical device”, is understood to be a wound dressing, a bandage, medical tubings, PU catheters and PU implants whereas “medical device surface” or “medical surface” is understood to be the surface of such devices.

In the context of the present patent application, “cross-linkable polymer adhesive” is understood to be either a “photo cross-linkable polymer adhesive” polymerizes under the exposure of UV light or a “non-photo cross-linkable polymer adhesive” that polymerizes independently of light of exposure.

The cross-linkable polymer adhesives of the present application have a low viscosity, improved adhesion to film, maintain tensile strength of film and biocompatible to human cells. A low viscosity of resin means the resin which can easily spread on the PU film. The improved adhesion means resin can strongly adhere to film after UV curing and does not leach or stick to other surfaces.

The present patent application describes, as the main embodiment of the invention, a process of coating a medical device surface comprising the steps of:
Preparation of a medical device surface;
Coating the medical device surface with a photo cross-linkable polymer adhesive;
Immobilization of peptide-based nanoparticles over the surface coated with the photo cross-linkable polymer adhesive;
Exposing the surface coated with the cross-linkable polymer adhesive and peptide-based nanoparticles to UV light with range of 365 o 395 nm;
Placing the medical device surface in phosphate buffer at a pH between 6 and 7.5 to leach loosely bound nanoparticles.

In one embodiment, the medical device surface is placed in the phosphate buffer for 120 to 360 min.

In one embodiment, the peptide-based nanoparticles are uniformly immobilized on the top of cross-linkable polymer adhesive coated medical devices after UV curing.

In one embodiment, the cross-linkable adhesive has viscosity between 3 and 300 cP.

In one embodiment, the UV light has a power of 100 W.

In one embodiment, the phosphate buffer has a molar concentration of 100 mM.

In one embodiment, the medical device surface comprises a film selected from polyurethane (PU), polystyrene (PS), poly(ethylene terephthalate) (PET) and polycarbonate (PC). In one preferable embodiment, the medical device is a wound dressing comprising a polyurethane (PU) film.

In one embodiment, the cross-linkable polymer adhesive is a photo cross-linkable adhesive.

In one preferable embodiment, the photo cross-linkable polymer adhesive comprise compounds selected from acrylated epoxies, acrylated polyesters, vinyl ethers, N-vinyl compound or vinylpyrrolidone compounds, wherein the said cross-linkable polymer adhesive polymerizes under the exposure of UV light.

In another embodiment, the cross-linkable polymer adhesive is a non-photo cross-linkable adhesive.

In one preferable embodiment, the non-photo cross-linkable adhesive is used to coat conjugated peptide-gold (Au) nanoparticles (LL37 NPs) on PU film wherein the said polymer adhesives are selected from dopamine, polyethylenimine, amino-propyltrimethoxy silane, polymer brushes containing trifluromethacrylate and 2hydroxyethyl methacrylate.

In one embodiment, the peptide-based nanoparticles are conjugated peptide-gold (Au) nanoparticles (LL37 NPs), wherein said peptide is LL37 (SEQ ID NO:1).

In one embodiment, the LL37 NPs are solubilized in ethanol, acetone, and dimethoxy sulfoxide (DMSO).

In one embodiment, LL37NPs are synthesized using LL37 peptide (0.1 to 0.25 mM) and HAuCl₄ (0.5 to 1 mM) in the presence of HEPES buffer (pH 5 and 7.5).

In another embodiment, the distance between UV light source and the film should be between 6 to 8 cm in order to coat LL37NPs.

In another embodiment, the amount of cross-linkable polymer adhesive should be 10 to 30 µL per cm² of film surface in order to have a very thin layer.

In one embodiment, the LL37 NPs have a concentration of 40 to 70 µg NPs/cm², preferably 60 µg NPs/cm².

According to the preferable embodiment of the present application, it is understood that, with the currently described approach, only a very negligible amount of LL37NPs ranging from 0.1 to 2 µg per cm² of PU-adhesive-LL37NP films leaches in PBS (pH 7.2) or in the wound environment.

Also part of the present application is the medical device obtained from the process described above, wherein said medical device comprises a medical device surface, a cross-linkable polymer adhesive and LL37 NPs, wherein said peptide is LL37 (SEQ ID NO: 1).

In one embodiment, the medical device surface comprises a film selected from polyurethane (PU), polystyrene (PS), poly(ethylene terephthalate) (PET) and polycarbonate (PC).

In one preferable embodiment, the medical device is a wound dressing comprising a polyurethane (PU) film.

In one embodiment, the cross-linkable polymer adhesive is a photo cross-linkable polymer adhesive or a non-photo cross-linkable polymer adhesive.

In one embodiment, photo cross-linkable polymer adhesive is selected from acrylated epoxies, acrylated polyesters, vinyl ethers, N-vinyl compound and vinylpyrrolidone compounds.

In another embodiment, the non-photo cross-linkable adhesive is selected from dopamine, polyethylenimine, amino-propyltrimethoxy silane, polymer brushes containing trifluromethacrylate and 2hydroxyethyl methacrylate.

In one embodiment, the top layer of the medical device is coated with LL37 NPs.

In one embodiment, the medical device has a water contact angle lower than 60°.

In one embodiment, the medical device surface comprises 10 to 30 µL per cm² of film surface.

In one embodiment, the LL37 NPs have a concentration of 40 to 70 µg NPs/cm².

According to the preferable embodiment of the present application, the medical device, preferably a wound dressing, has a water contact angle lower than 60° wherein the said contact angle should be hydrophilic in order to maintain moist environment of wound.

According to the preferable embodiment of the present application, the medical device, preferably the PU-adhesive-LL37NP films of the application, kill Gram-positive and Gram-negative bacteria from 1 log to 4 log.

According to the preferable embodiment of the present application, the medical device, preferably the PU-adhesive-LL37NP films of the application, kills bacteria without inducing resistance in sub-MIC concentrations of the immobilized peptide.

According to the preferable embodiment of the present application, the medical device, preferably the PU-adhesive-LL37NP films of the application, promote rapid healing of diabetic wounds by the expression of keratin 14 and 5 along with transition of early macrophages (M1) to late macrophages (M2) in day 6 wounds.

This description is of course not in any way restricted to the forms of implementation presented herein and any person with an average knowledge of the area can provide many possibilities for modification thereof without departing from the general idea as defined by the claims. The preferred forms of implementation described above can obviously be combined with each other. The following claims further define the preferred forms of implementation.

1. A process of coating a medical device surface comprising the steps of:
preparing a medical device surface;
coating the medical device surface with a photo cross-linkable polymer adhesive;
immobilizing peptide-based nanoparticles over the top of the surface coated with the cross-linkable polymer adhesive after UV curing;
exposing the surface coated with the cross-linkable polymer adhesive and peptide-based nanoparticles to an UV light source with wavelength of from 365 to 395 nm;
placing the medical device surface in phosphate buffer at a pH between 6 and 7.5 to leach loosely bound nanoparticles.

2. The process of coating a medical device according to claim 1, wherein the medical device surface is placed in the phosphate buffer for 120 to 360 minutes.

3. The process of coating a medical device according claim 1, wherein the cross-linkable polymer adhesive has a viscosity between 3 and 300 cP.

4. The process of coating a medical device according to claim 1, wherein the medical device surface comprises a film selected from polyurethane (PU), polystyrene (PS), poly(ethylene terephthalate) (PET) and polycarbonate (PC).

5. The process of coating a medical device according to claim 1, wherein the medical device is a wound dressing comprising a polyurethane (PU) film.

6. The process of coating a medical device according to claim 1, wherein the cross-linkable polymer adhesive is a photo cross-linkable adhesive.

7. The process of coating a medical device according to claim 1, wherein the photo cross-linkable polymer adhesive is selected from acrylated epoxies, acrylated polyesters, vinyl ethers, N-vinyl compound and vinylpyrrolidone compounds.

8. The process of coating a medical device according to claim 1, wherein the cross-linkable polymer adhesive is a non-photo cross-linkable adhesive.

9. The process of coating a medical device according to claim 1, wherein the non-photo cross-linkable polymer adhesive is selected from the group consisting of dopamine, polyethylenimine, amino-propyltrimethoxy silane, polymer brushes containing trifluromethacrylate and 2hydroxyethyl methacrylate.

10. The process of coating a medical device according to claim 1, wherein the peptide-based nanoparticles are LL37 NPs, wherein said peptide is LL37 (SEQ ID NO:1).

11. The process of coating a medical device according to claim 1, wherein the LL37 NPs are solubilized in ethanol, acetone, and dimethoxy sulfoxide (DMSO).

12. The process of coating a medical device according to claim 1, wherein the distance between UV light source and the film should be between 6 to 8 cm in order to coat LL37NPs.

13. The process of coating a medical device according to claim 1, wherein the amount of polymer adhesive should be between 10 to 30 µL per cm² of film surface.

14. The process of coating a medical device according to claim 1, wherein the LL37 NPs have a concentration of 40 to 70 µg NPs/cm².

15. The process of coating a medical device according to claim 1, wherein the amount of peptide available on the surface of medical device should be between 13 to 23 µg/cm².

16. A medical device comprising a medical device surface, a photo cross-linkable polymer adhesive, a cross-linkable polymer adhesive and LL37 NPs, wherein said peptide is LL37 (SEQ ID NO: 1).

17. The medical device according to claim 16, wherein the cross-linkable polymer adhesive has a viscosity of between 3 and 300 cP.

18. The medical device according to claim 16, wherein the medical device surface comprises a film selected from polyurethane (PU), polystyrene (PS), poly(ethylene terephthalate) (PET) and polycarbonate (PC).

19. The medical device according to claim 16, wherein the medical device is a wound dressing comprising a polyurethane (PU) film.

20. The medical device according to claim 16, wherein the cross-linkable polymer adhesive is a photo cross-linkable polymer adhesive.

21. The medical device according to claim 16, wherein the photo cross-linkable polymer adhesive is selected from acrylated epoxies, acrylated polyesters, vinyl ethers, N-vinyl compound and vinylpyrrolidone compounds.

22. The medical device according to claim 16, wherein the cross-linkable polymer adhesive is a non-photo cross-linkable polymer adhesive.

23. The medical device according to claim 16, wherein the non-photo cross-linkable polymer adhesive is selected from dopamine, polyethylenimine, amino-propyltrimethoxy silane, polymer brushes containing trifluromethacrylate and 2hydroxyethyl methacrylate.

24. The medical device according to claim 16, wherein the LL37 NPs are immobilized on the top of the cross-linkable polymer adhesive coating the medical device.

25. The medical device according to claim 16, wherein, the medical device has a water contact angle lower than 60°.

26. The medical device according to claim 16, wherein the medical device surface comprises 10 to 30 µL per cm² of film surface.

27. The medical device according to claim 16, wherein the LL37 NPs have a concentration of 40 to 70 µg NPs/cm².


SEQUENCE LISTING (9 sequences)

SEQ ID NO: 1 - protein, 38 aa, Artificial Sequence
  Cathelicidin Antimicrobial Peptide LL-37 Modified with C-terminal cysteine
LLGDFFRKSKEKIGKEFKRIVQRIKDFLRNLVPRTESC

SEQ ID NO: 2 - DNA, 20 bp, Artificial Sequence
  GADPH F primer for qRT-PCR analysis
agccacatcgctcagacacc

SEQ ID NO: 3 - DNA, 19 bp, Artificial Sequence
  GADPH R primer for qRT-PCR analysis
gtactcagcgccagcatcg

SEQ ID NO: 4 - DNA, 18 bp, Artificial Sequence
  TNF-alfa F primer for qRT-PCR analysis
gtctcagcctcttctatt

SEQ ID NO: 5 - DNA, 20 bp, Artificial Sequence
  TNF-alfa R primer for qRT-PCR analysis
ccatttgggaacttctcatc

SEQ ID NO: 6 - DNA, 20 bp, Artificial Sequence
  IL-6 F primer for qRT-PCR analysis
acctgtctataccacttcac

SEQ ID NO: 7 - DNA, 23 bp, Artificial Sequence
  IL-6 R primer for qRT-PCR analysis
ggcaaatttcctgattatatcca

SEQ ID NO: 8 - DNA, 20 bp, Artificial Sequence
  IL-10 F primer for qRT-PCR analysis
cacaaagcagccttgcagaa

SEQ ID NO: 9 - DNA, 21 bp, Artificial Sequence
  IL-10 R primer for qRT-PCR analysis
agagcaggcagcatagcagtg